(19) United States
(12) Patent Application Publication
(10) Pub. No.: US 2022/0331176 A1
(43) Pub. Date: Oct. 20, 2022

(54) A HYGIENE ARTICLE

(71) Applicant: Woolchemy NZ Limited, Upper Hutt (NZ)

(72) Inventors: Alistair ROGERS, Upper Hutt (NZ); Manoj RATHOD, Upper Hutt (NZ); Steven NEILL, Upper Hutt (NZ); Fadi JUNAID, Upper Hutt (NZ); Derelee POTROZ-SMITH, Upper Hutt (NZ)

(21) Appl. No.: 17/754,355

(22) PCT Filed: Oct. 2, 2020
(86) PCT No.: PCT/NZ2020/050120

§ 371 Date: Mar. 30, 2022

(30) Foreign Application Priority Data
Oct. 4, 2019 (NZ) 757820

(51) Int. Cl. A61F 13/537; A61F 13/15; D04H 1/4258; D04H 1/4266; D04H 1/492; D04H 1/541; D04H 1/55; D04H 1/58

(52) U.S. Cl. CPC A61F 13/537; A61F 13/15252; D04H 1/4258; D04H 1/4266; D04H 1/492; D04H 1/5412; D04H 1/55; D04H 1/58; A61F 2013/530386

(57) ABSTRACT

The present invention is directed to a hygiene article for use in personal care absorbent products such as nappies or diapers, training pants, sanitary napkins, incontinence garments, wound aids, personal protective equipment, facemasks, gowns, head and shoe covers and the like. More particularly, invention provides a hygiene article including a fibre composition comprising a combination of sheep wool fibres and polymeric fibres, wherein the polymeric fibres are not derived from one or more petrochemicals.

### FIELD OF THE INVENTION

The present invention is directed to a hygiene article for use in personal care absorbent products such as nappies or diapers, training pants, sanitary napkins, incontinence garments, wound aids, personal protective equipment, facemasks, gowns, head and shoe covers and the like. More particularly, the hygiene article is a nonwoven sheep wool containing hygiene article.

### BACKGROUND OF THE INVENTION

A variety of hygiene products are available on the market. Many of these products include materials that are not renewable or sustainably produced, such as absorbent polymeric materials derived from virgin petrochemicals.

Sheep wool has been used in durable products that are washable and/or hard wearing, such as garments, furnishings such as carpets and furniture upholstery and insulating building materials. Wool has innate properties such as thermal regulation, odour control, breathability, and comfort. In light of environmental concerns in fast moving consumer products, wool has the ability to be used to fill a gap in sustainable single-use hygienic non-woven products or articles.

It is an object of the present invention to provide a renewable and sustainably produced hygiene article comprised from materials that are not derived from petrochemicals, or to at least provide the public with an environmentally responsible and useful alternative.

### SUMMARY OF THE INVENTION

In a first aspect the present invention provides a hygiene article including a fibre composition comprising a combination of sheep wool fibres and polymeric fibres, wherein the polymeric fibres are not derived from one or more petrochemicals.

In one embodiment the hygiene article is a non-woven product.

In one embodiment the polymeric fibres are sustainably produced and biodegradable or compostable.

In one embodiment the sheep wool fibres are scoured wool fibres. In one embodiment the sheep wool fibres are between about 16-40 microns in thickness. In another embodiment the wool fibres are between about 18-40 microns in thickness. In another embodiment the wool fibres are between about 30-40 microns in thickness.

In one embodiment the wool fibre length is between about 25-130 mm. In one embodiment the wool fibre length is between about 25-75 mm.

In one embodiment the hygiene article is configured as an acquisition distribution layer, a filter layer, a moisture resistance textile, an antimicrobial textile, a topsheet or an absorbent core layer. In one embodiment the hygiene article is configured as an acquisition distribution layer. In one embodiment the hygiene article is configured as a filter layer suitable for use in a facemask or the like. In one embodiment the hygiene article is configured as a moisture resistance textile suitable for use in personal protective wearable equipment, such as but not limited to gowns, shoe coverings, head coverings or the like. In one embodiment the hygiene article is configured as an antimicrobial textile for use in personal protective wearable equipment, such as but not limited to gowns, shoe coverings, head coverings or the like.

In one embodiment the fibre composition comprises between about 20-99% wool fibre and between about 1-80% polymeric fibres. In a preferred embodiment the fibre composition comprises between about 20-97% wool fibres and between about 3-80% polymeric fibres. In a more preferred embodiment, the fibres composition comprises about 30-95% wool fibres and about 5-70% polymeric fibres.

In one embodiment the fibre composition may further include up to 25% of a fibre binder.

In one embodiment the polymeric fibres are derived from but not limited to viscose, rayon, bagasse, hemp, pineapple plant fibres, flax, cotton, eucalyptus, silk, sugar cane, corn starch, soybean, rice waste, recycled fibres, milk fibres, recovered fibres and the like. In one embodiment the polymeric fibres are selected from polylactic acid fibres, cellulosic polymeric fibres or combinations thereof.

In another embodiment the polymeric fibres are between about 30-40 mm in length.

In one embodiment the fibre composition comprises between about 20-70% wool fibre and between about 80-30% polylactic acid fibres. In a preferred embodiment the fibre composition comprises between about 40-60% wool fibres and between about 60-40% polylactic acid fibres. In a more preferred embodiment the fibres composition comprises between about 45-55% wool fibres and between about 55-45% polylactic acid fibres. In a most preferred embodiment the fibre composition comprises between about 50% wool fibres and about 50% polylactic acid fibres.

In one embodiment the fibre composition comprises between about 20-70% wool fibre and between about 80-30% cellulosic fibres. In a preferred embodiment the fibre composition comprises between about 30-45% wool fibres and between about 30-45% cellulosic fibres and between about 10-40% of a fibre binder.

In one embodiment the cellulosic fibres are between about 30-40 mm in length.

In one embodiment the cellulosic fibres are viscose fibres.

In another embodiment the fibre composition comprises between about 20-99% wool fibre and between about 1-80% viscose fibres. In another embodiment the fibre composition comprises between about 80-98% wool fibres and between about 2-20% viscose fibres. In a more preferred embodiment the fibres composition comprises between about 90-96% wool fibres and between about 4-10% viscose fibres. In a most preferred embodiment the fibre composition comprises about 95% wool fibres and about 5% viscose fibres.

In one embodiment the viscose fibres are between about 30-40 mm in length.

In one embodiment the weight of the hygiene article is between about 30-200 gram per square meter. In a preferred embodiment the weight of the hygiene article is about 50-100 gram per square meter. In another preferred embodiment the weight of the hygiene article is about 60 gram per square meter.

In one embodiment the thickness of the hygiene article is between about 0.5 to 2.0 mm. In a preferred embodiment the thickness of the hygiene article is between about 1.0 to 1.5 mm. In a more preferred embodiment the thickness of the hygiene article is about 1.0 mm.

In one embodiment the hygiene article is configured as an acquisition distribution layer, a filter layer, a moisture resistance textile, an antimicrobial textile, a topsheet or an absorbent core layer. In one embodiment the hygiene article is configured as an acquisition distribution layer. In one embodiment the hygiene article is configured as a filter layer suitable for use in a facemask or the like. In one embodiment the hygiene article is configured as a moisture resistance textile suitable for use in personal protective wearable equipment, such as but not limited to gowns, shoe coverings, head coverings or the like. In one embodiment the hygiene article is configured as an antimicrobial textile for use in personal protective wearable equipment, such as but not limited to gowns, shoe coverings, head coverings or the like.

In a second aspect the present invention provides a method of producing a hygiene article, as defined above, the method including the steps of:
(a) combining wool fibres and polymeric fibres together to provide a fibre composition; and
(b) bonding the combined fibres.

In one embodiment the method includes the further step of applying a wetting agent to the fibre composition either before or after the bonding step. In one embodiment the wetting agent is Cirrasol 910XS-LQ (CRODA).

In one embodiment the hygiene article is an acquisition distribution layer, a topsheet or an absorbent core layer. In one embodiment the hygiene article is an acquisition distribution layer.

In one embodiment the step of combining the wool fibres and polymeric fibres together to provide a fibre composition is achieved by a dry laid technique (eg carding), an air laid technique or a wet laid technique.

In one embodiment the method includes the further step of drying the fibre composition after the wetting agent has been applied.

In one embodiment the bonding step is selected from a thermal bonding step, a mechanical bonding step or a chemical bonding step. In one embodiment the mechanical bonding step is a hydroentanglement step or a spunlacing step or an air laid step.

In one embodiment the bonding step is a thermal bonding step carried out at about 130 degrees C. In one embodiment, the thermal bonding step is carried out for about 1 minute.

In one embodiment the hygiene article is a non-woven product.

In a third aspect the present invention provides a method of producing a hygiene article, as defined above, the method including the steps of:
(a) combining wool fibres and polymeric fibres together to provide a fibre composition; and
(b) spunlacing the combined fibres.

In one embodiment the step of combining the wool fibres and polymeric fibres together to provide a fibre composition is achieved by a dry laid technique (eg carding), an air laid technique or a wet laid technique. In one embodiment the fibre composition is formed into a web formation.

In one embodiment the spunlacing step is carried out at between about 70-100 bar.

In one embodiment the spunlacing step is carried out with 2 passes.

In one embodiment the hygiene article is an acquisition distribution layer, a topsheet or an absorbent core layer. In one embodiment the hygiene article is an acquisition distribution layer.

In one embodiment the hygiene article is a non-woven product.

Further aspects and embodiments of the invention will become apparent from the following description and examples that are provided below.

### DESCRIPTION OF EMBODIMENTS OF THE INVENTION

The invention is now disclosed in more detail below, with reference to the Figures and Examples that are provided in a non-limiting manner.

### BRIEF DESCRIPTION OF THE FIGURES

FIG. 1: represents a schematic illustration of the top view of a hygiene article, specifically an acquisition distribution layer located in a diaper or a sanitary pad

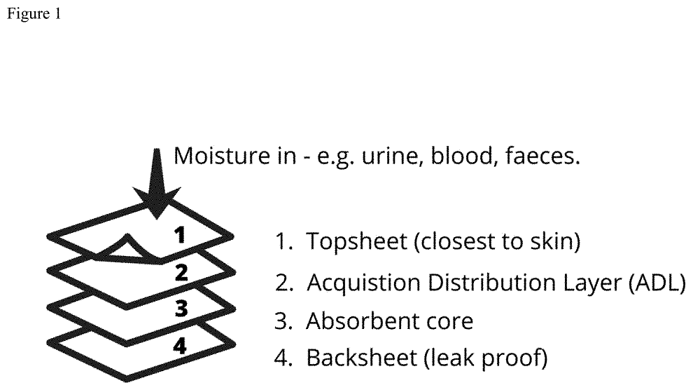

FIG. 2: shows a photo of a hygiene article, by way of example an acquisition distribution layer, rolled into a format for use in diaper manufacture.

FIG. 3: shows a photo of examples of nonwoven hygiene articles. The green article shows a nonwoven acquisition distribution article made from synthetic fibres. The white nonwoven articles are bio-composite nonwovens comprising wool and viscose and wool and polylactic acid (PLA).

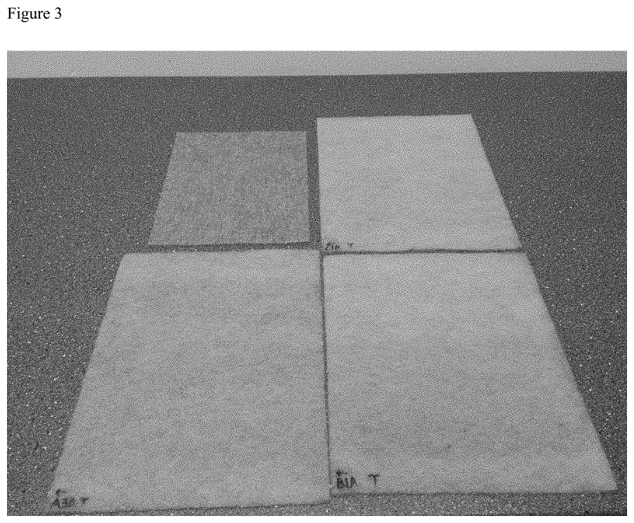

FIG. 4: represents an overall view of one embodiment of the process of the invention for the manufacture of a non-woven hygiene article according to the invention.

The following description sets forth numerous exemplary configurations, parameters, and the like. It should be recognised, however, that such description is not intended as a limitation on the scope of the present invention, but is instead provided as a description of exemplary embodiments.

### Definitions

In each instance herein, in descriptions, embodiments, and examples of the present invention, the terms “comprising”, “including”, etc., are to be read expansively, without limitation.

Thus, unless the context clearly requires otherwise, throughout the description and the claims, the words “comprise”, “comprising”, and the like are to be construed in an inclusive sense as to opposed to an exclusive sense, that is to say in the sense of “including but not limited to”. As used herein, the terms “about” or “approximately” usually means within 20%, more preferably within 10%, and most preferably still within 5% of a given value or range.

Alternatively, the term “about” means within a log (i.e., an order of magnitude) preferably within a factor of two of a given value.

As used herein, the term “article” refers to any three-dimensional solid material being able to acquire and store body fluids discharged from the body. Preferred articles according to the present invention are disposable fluid-absorbent articles that are designed to be worn in contact with the body of a user such as facemasks, personal protection wearable equipment such as but not limited to gowns, shoe coverings, head coverings or the like, disposable fluid-absorbent pantiliners, sanitary napkins, catamenials, incontinence inserts/pads, diapers, training pant diapers, breast pads, interlabial inserts/pads and the like.

As used herein, the term “body fluids” refers to any fluid produced and discharged by human or animal body, such as urine, menstrual fluids, faeces, vaginal secretions and the like.

As user herein, the term “cellulosic polymeric fibres” refers to any naturally produced or mechanically produced fibres derived from a cellulosic source, and includes but is not limited to cotton, wood or wood pulp, linen, hemp, bagasse, flax, eucalyptus, sugar cane, corn starch, soybean, rice waste, rayon, viscose, jute fibres, pineapple plant fibres, recovered and recycled fibres and combinations thereof.

As used herein, the term “layer” refers to a composition of fibres whose primary dimension is along its length and width. It should be known that the term “layer” is not necessarily limited to single layers or sheets of the composition. Thus a layer can comprise laminates, composites, combinations of several sheets or webs of different fibrous materials.

### EXAMPLES

The example described herein is provided for the purpose of illustrating specific embodiments of the invention and is not intended to limit the invention in any way. Persons of ordinary skill can utilise the disclosures and teachings herein to produce other embodiments and variations without undue experimentation. All such embodiments and variations are considered to be part of this invention.

Technologies for consolidating fibers or bonding the fibres in a layer or web are mechanical bonding, thermal bonding and chemical bonding. In the process of mechanical bonding the fibers are entangled mechanically, e.g., by water jets (spunlace) or barbed needles (needle punch) to entangle and fuse the fibres together and give integrity to the web. Thermal bonding is carried out by means of raising the temperature in the presence of low-melting thermoplastic like polylactic acid that melts at a specific temperature to bond fibres within the layer or web. Chemical bonding is carried out by using a wet chemical binder to bind the fibres in the web formation.

Preferred means of increasing the integrity are thermal bonding, needle punching, through-air bonding and/or spunlacing. Upon wetting, the structure and integrity of the hygiene article remains stable. In summary, the addition of thermoplastic material leads to improved fluid permeability of discharged body fluids and thus to improved acquisition properties.

The preferred thermal bonding technology is achieved through the application of hot air to the surface of the fibrous fabric. The hot air is circulated just above the fibrous fabric, but does not push through the fibrous fabric. Bonding sites are generated between the wool fibres and the polymeric fibres. It is envisioned that it may be appropriate to use suitable binders during the thermal bonding process such as crystalline binder fibers, bi-component binder fibers, and powders. When using crystalline binder fibers or powders, the binder melts entirely and forms molten droplets throughout the nonwoven's cross-section. Bonding occurs at these points upon cooling. Products manufactured using through-air ovens tend to be bulky, open, soft, strong, extensible, breathable and absorbent.

In the case of thermal bonding, a thermoplastic material may also be added to the fibers. Upon thermal treatment at least a portion of this thermoplastic material is melting and migrates to intersections of the fibers caused by capillary effects. These intersections solidify to bond sites after cooling and increase the integrity of the fibrous matrix.

Suitable thermoplastic materials include polylactic acid and any other biodegradable thermosensitve polymer.

Spunlacing (also known as “hydroentanglement”) is a further method of increasing the integrity of a web. The formed web of carded or loose fibers (usually air-laid or wet-laid) is first compacted and prewetted to eliminate air pockets. The technology of spunlacing uses multiple rows of fine high-speed jets of water to strike the web on a porous belt or moving perforated or patterned screen so that the fibers knot about one another. The water pressure generally increases from the first to the last injectors. Pressures as high as 150 bar are used to direct the water jets onto the web. This pressure is sufficient for most of the nonwoven fibers, although higher pressures are used in specialized applications.

The spunlace process is a nonwovens manufacturing system that employs jets of water to entangle fibers and thereby provide fabric integrity. Softness, drape, conformability, and relatively high strength are the major characteristics of spunlace nonwoven.

Chemical bonding is carried out by using a chemical binder to bind the fibres in a web formation. A chemical binder agent, such as a binder (OC-BioBinder™ Oak 33XX from OrganoClick) may be applied during the chemical bonding step. The chemical bonding step may involve a continuous process of moving the fibred web formation through an impregnation bath or sprayer containing the binder. It is to be appreciated that variations to the chemical bonding techniques can be employed, such as adding a wetting agent to the impregnation bath containing the binder. The time for chemical bonding needs to be sufficient for impregnation of the fibres to occur to enable the fibres to form a chemical bond with the binder agent to thereby enhance the physical integrity of the fibre composition produced.

### Hygiene Article

A hygiene article of the invention, such as but not limited to an acquisition distribution layer, is preferably constructed to efficiently transfer and distribute discharged body fluids to other regions of the composition or to other layers, where the body fluids are immobilized and stored.

The hygiene article comprises wool fibres and polymeric fibres.

The wool fibres may be fixed to increase the strength and the integrity of the article. Technologies for consolidating fibers in a web are mechanical bonding, thermal bonding and chemical bonding. A preferred hygiene article comprises wool fibres and polymer fibres distributed within.

Thus, by way of example, a suitable hygiene article would comprise from 50 to 99% by weight wool material and from 1 to 50% by weight polymer fibres; preferably from 50 to 80% by weight wool material and from 20 to 50% by weight polymer fibres.

If the hygiene article is configured as an acquisition distribution layer, the weight range of the article would be from 20 to 200 g/m², most preferred in the range from 40 to 80 g/m², depending on the percentage of polymer fibres.

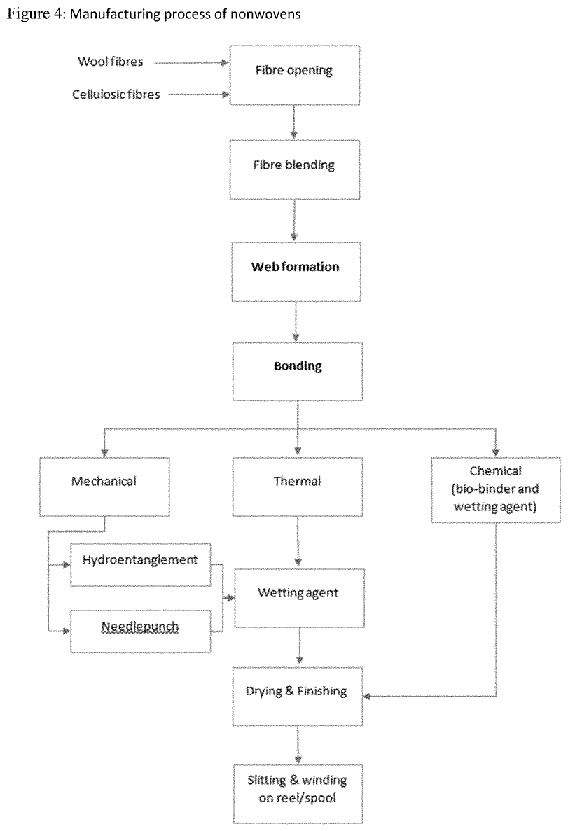

The process for making a hygiene article of the invention is generally outlined in FIG. 4. The process involves combining scoured raw wool and polymeric fibres in a ratio, such as 50% wool fibres and 50% polylactic acid fibres and forming those fibres together into a web formation. The fibres are then bonded (either thermally, chemically or mechanically) to effect a physical connection or bond between the polymeric fibres and the wool fibres and to increase the integrity of the composition. Before or after the bonding step, a wetting agent may be applied to the resulting wool/polymeric fibre composition. The wetting step is undertaken to make the wool fibres hydrophilic. If wetting agent is applied to the fibre composition, then the fibre composition will require further drying to dry off the water applied during application of the wetting agent step. The final step requires the spooling of the fibre composition

The following examples illustrate the invention without limiting it.

### Example 1

A first fibre composition was produced using 50% scoured New Zealand wool having a fibre length of about 25-130 mm a wool diameter of between about 18 to about 40 microns, preferably about 34-39 microns and 50% polylactic acid (PLA) bicomponent 2.2 dtex and having a fibre length of about 38 mm. The PLA was sourced directly from Ingeo Fibres. The separated fibres were layered up and blended using a Frenaught opener machine. The fibres were carded together using a carding machine (0.5 m Single Cylinder Tatham Card) in a two pass process. The first pass blended fibres through card to further blend the fibres. In the second pass the carded fibred web was rotated 90 degrees to help re-orientate the fibres in the second carding pass in order to produce a uniform and homogenous web. The resulting carded fibre web was then thermally bonded using a hot press at approximately 140 degrees C. for approximately 20 secs in total (10 seconds on one side, sample then turned over and then pressed again for a further 10 seconds). It is to be appreciated that alternative thermal bonding techniques can be used, such as a flat-be laminator or a through air oven with top and bottom conveyers. The time for thermal bonding needs to be sufficient for the PLA to heat to allow flowing of the sheath of the PLA fibre and then effectively a cooling period to allow the fibre composition to set before any spooling step. Prior to spooling the wool and PLA fibre composition is optionally wetted using a wetting agent to make the wool hydrophilic. In this example, a wetting agent was used, specifically a 1% solution (diluted with water) of Cirrasol 910XS-LQ (sourced from CRODA International Plc). After the wetting agent was applied the fibre composition was subjected to a drying step using a Spooner through-air oven at about 80 degrees C. and at 50% fan speed for around 2 minutes or long enough to remove the water. Higher temperatures may be used in the drying step, however, the temperature must not affect the integrity of the fibre composition. Once the fibre composition had been prepared it was subjected to various tests to determine the properties of the fibre composition as outlined below. The tests were conducted in accordance with the non-woven standards procedures.

### Results:

The results of the acquisition distribution layer produced according to Example 1 as described above are tabulated below in Table 1 and the results are also compared to a commercial acquisition distribution layer—see Table 2.

TABLE 1
Properties of Example 1

| Specification | Test Standard | Average | Std Dev | Units |
|---|---|---|---|---|
| Weight | WSP 130.1.RO (15) | 56.55 | 2.16 | g · m⁻² |
| Thickness | WSP 120.6.RO (15) | 1.25 | 0.21 | mm |
| Rewet | WSP 080.10.RO (15) | 0.1067 | 0.0156 | g |
| Strike-Through | WSP 070.3.RO (15) | 1.84 | 0.07 | second |
| time | | | | |
| Tenacity (CRE) | WSP 110.4.RO (15) | 23.38 | 2.00 | N/2.5 cm |
| Opt A | | | | |
| Elongation (CRE) | WSP 110.4.RO (15) | 28.46 | 1.31 | % |
| Opt A | | | | |

TABLE 2
Commercial acquisition layer comparator having a fibre composition
of polyester staple and bicomponent (PES and BICO)

| Specification | Test Standard | Average | Std Dev | Units |
|---|---|---|---|---|
| Weight | WSP 130.1.RO (15) | 60.6 | — | g · m⁻² |
| Thickness | WSP 120.6.RO (15) | 1.88 | 0.05 | mm |
| Rewet | WSP 080.10.RO (15) | 0.0980 | 0.0100 | g |
| Strike-Through | WSP 070.3.RO (15) | 1.80 | 0.06 | second |
| time | | | | |
| Tenacity (CRE) | WSP 110.4.RO (15) | 28.40 | 2.17 | N/2.5 cm |
| Opt A | | | | |
| Elongation (CRE) | WSP 110.4.RO (15) | 16.9 | 1.91 | % |
| Opt A | | | | |

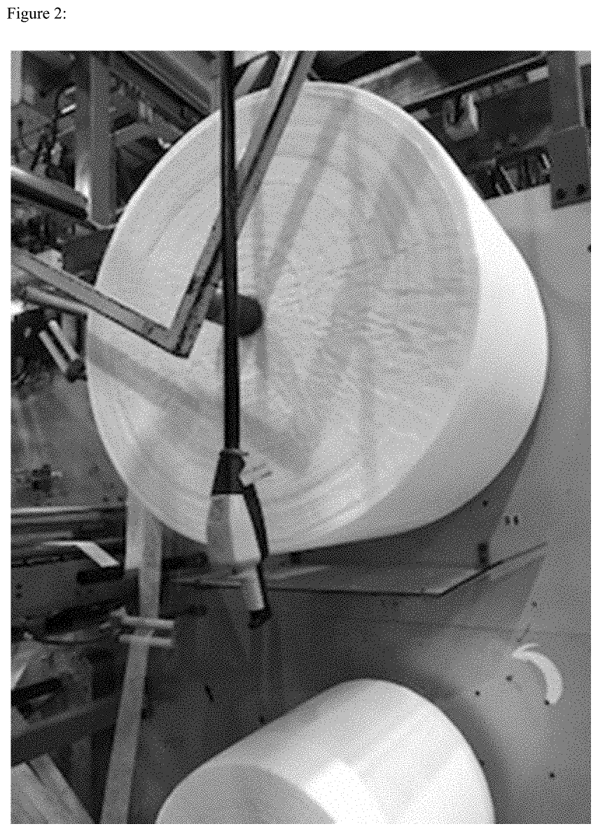

It can be seen from the attached that the strike through and rewet properties of the acquisition distribution layer produced according to Example 1 and the process outlined in FIG. 2 compare well to the rewet and strikethrough properties of a commercial acquisition distribution layer.

### Example 2

A second fibre composition was produced using 40% scoured New Zealand wool having a fibre length of about 25-130 mm, a wool fibre diameter of between about 20 to about 40 microns, preferably about 34-39 microns and 40% viscose (1.3 dtex) and having a fibre length of about 38 mm. The viscose fibre was sourced directly from Lenzing. The separated fibres were layered up and blended using a Frenaught opener machine. The fibres were carded together using a carding machine (0.5 m Single Cylinder Tatham Card) in a two pass process. The first pass blended fibres through card to further blend the fibres. In the second pass the carded fibred web was rotated 90 degrees to help re-orientate the fibres in the second carding pass in order to produce a uniform and homogenous web. The binder was applied before the thermal bonding step. The resulting fibre web was then thermally bonded using a hot press at approximately 140 degrees C. for approximately 20 secs in total (10 seconds on one side, sample then turned over and then pressed again for a further 10 seconds). It is to be appreciated that alternative thermal bonding techniques can be used, such as a flat-be laminator or a through air oven with top and bottom conveyers. The time for thermal bonding needs to be sufficient for the viscose to heat to allow flowing of the sheath of the viscose fibre and then effectively a cooling period to allow the fibre composition to set before any further step. Prior to spooling the wool and viscose fibre composition is optionally wetted using a wetting agent to make the wool hydrophilic. In this example, a wetting agent was used, specifically a 1% solution (diluted with water) of Cirrasol 910XS-LQ (sourced from CRODA International Plc). After the wetting agent was applied the fibre composition was subjected to a drying step using a Spooner through-air oven at about 80 degrees C. and at 50% fan speed for around 2 minutes or long enough to remove the water. Higher temperatures may be used in the drying step, however, the temperature must not affect the integrity of the fibre composition. Once the drying step had been completed a binder was applied to the fibre composition to make up the other 20% of the composition. The binder, OC-BioBinder Oak was sourced from OrganoClick. The binder was applied using a one pass padding process at 1 bar pressure and at 1 m/min (Roaches pad mangle). A subsequent drying step using a one pass through the air oven at 100 degrees C. for 2 mins at 40% fan speed was employed. Once the fibre composition had been prepared it was subjected to various tests to determine the properties of the fibre composition as outlined below. The tests were conducted in accordance with the NonWoven Standards Procedures.

### Results:

The results of the acquisition distribution layer produced according to Example 2 as described above are tabulated below in Table 3.

TABLE 3
Properties of Example 2

| Specification | Test Standard | Average | St Dev | Units |
|---|---|---|---|---|
| Weight | WSP 130.1.RO (15) | 61.10 | 2.24 | g · m⁻² |
| Thickness | WSP 120.6.RO (15) | 0.86 | 0.11 | mm |
| Rewet | WSP 080.10.RO (15) | 2.7281 | 1.1647 | g |
| Strike-Through | WSP 070.3.RO (15) | 2.29 | 0.25 | second |
| time | | | | |
| Tenacity (CRE) | WSP 110.4.RO (15) | 23.76 | 2.20 | N/2.5 cm |
| Opt A | | | | |
| Elongation (CRE) | WSP 110.4.RO (15) | 11.73 | 0.73 | % |
| Opt A | | | | |

The results show that the increase in surface area and wettability due to viscose fibre content helped improve the tensile and elongation properties, (comparable to the properties measured for the commercial sample as tabulated in Table 2), however, the rewet and strike through results were negatively affected with the viscose fibre content at 40%.

### Example 3

A third fibre composition was produced using 95% scoured New Zealand wool having a fibre length of about 25 to 130 mm and 5% viscose (1.3 dtex) and having a fibre length of about 38 mm. The viscose fibre was sourced directly from Lenzing. The separated fibres were layered up and blended using a Frenaught opener machine. The fibres were carded together using a carding machine (0.5 m Single Cylinder Tatham Card) in a two pass process. The first pass blended fibres through card to further blend the fibres. In the second pass the carded fibred web was rotated 90 degrees to help re-orientate the fibres in the second carding pass in order to produce a uniform and homogenous web. The resulting fibre web was then spun lace/hydroentangled at approximately 70 bar over two passes for approximately 20 secs in total (10 seconds on one side, sample then turned over and then hydroentangled again for a further 10 seconds). After the hydroentanglement step, the fibre composition was subjected to a drying step using a Spooner through-air oven at about 80 degrees C. and at 50% fan speed for around 2 minutes or long enough to remove the water. Higher temperatures may be used in the drying step, however, the temperature must not affect the integrity of the fibre composition. Once the fibre composition had been prepared it was subjected to various tests to determine the properties of the fibre composition as outlined below. The tests were conducted in accordance with the NonWoven Standards Procedures and compared to the Properties of the Commercial Product—see Table 2.

TABLE 4
Properties of Example 3

| Specification | Test Standard | Average | St Dev | Units |
|---|---|---|---|---|
| Weight | WSP 130.1.RO (15) | 78.91 | — | g · m⁻² |
| Thickness | WSP 120.6.RO (15) | 0.95 | 0.04 | mm |
| Rewet | WSP 080.10.RO (15) | 0.147 | 0.051 | g |
| Strike-Through | WSP 070.3.RO (15) | 1.64 | 0.13 | second |
| time | | | | |
| Tenacity (CRE) | WSP 110.4.RO (15) | 14.25 | 2.97 | N/2.5 cm |
| Opt A | | | | |
| Elongation (CRE) | WSP 110.4.RO (15) | 81.20 | 4.02 | % |
| Opt A | | | | |

### Example 4

A fourth fibre composition was produced using 60% scoured New Zealand wool having a fibre length of about 25 to 130 mm, 15% cotton, 5% viscose (1.3 dtex) and 20% biobinder. The cotton and viscose had a fibre length of about 38 mm. The viscose fibre was sourced directly from Lenzing. The cotton fibre was sourced from TJ Beall Co. The separated fibres were layered up and blended using a Frenaught opener machine. The fibres were carded together using a carding machine (0.5 m Single Cylinder Tatham Card) in a two pass process. The first pass blended fibres through card to further blend the fibres. In the second pass the carded fibred web was rotated 90 degrees to help re-orientate the fibres in the second carding pass in order to produce a uniform and homogenous web. The first pass blended fibres through card to further blend the fibres. In the second pass the carded fibred web was rotated 90 degrees to help re-orientate the fibres in the second carding pass in order to produce a uniform and homogenous web. The binder (OC-BioBinder™ Oak 33XX from Organ® Click) was applied during the chemical bonding step. The resulting fibre web was then chemically bonded. The chemical bonding step involved a continuous process of moving the fibred web formation through an impregnation bath or sprayer containing the binder. It is to be appreciated that variations to the chemical bonding techniques can be employed, such as adding a wetting agent to the impregnation bath containing the binder. The time for chemical bonding needs to be sufficient for impregnation of the fibres to occur. Prior to spooling the wool, cotton and viscose fibre composition is dried. Once the fibre composition had been prepared it was subjected to various tests to determine the properties of the fibre composition as outlined below. Two slightly different weighted samples (Sample 1 and Sample 2) were prepared using identical processes. The tests were conducted in accordance with the non-woven standards procedures and compared to the Properties of the Commercial Product—see Tables 5 and 6.

TABLE 5
Properties of Example 4 (sample 1)

| Specification | Test Standard | Average | St Dev | Units |
|---|---|---|---|---|
| Weight | WSP 130.1.RO (15) | 71.65 | — | g · m⁻² |
| Thickness | WSP 120.6.RO (15) | 1.59 | 0.14 | mm |
| Rewet | WSP 080.10.RO (15) | 0.146 | 0.035 | g |
| Strike-Through | WSP 070.3.RO (15) | 1.79 | 0.14 | second |
| time | | | | |
| Tenacity (CRE) | WSP 110.4.RO (15) | 7.49 | 0.74 | N/2.5 cm |
| Opt A | | | | |
| Elongation (CRE) | WSP 110.4.RO (15) | 8.65 | 1.40 | % |
| Opt A | | | | |

TABLE 6
Properties of Example 4 (sample 2)

| Specification | Test Standard | Average | St Dev | Units |
|---|---|---|---|---|
| Weight | WSP 130.1.RO (15) | 61.60 | — | g · m⁻² |
| Thickness | WSP 120.6.RO (15) | 1.20 | 0.10 | mm |
| Rewet | WSP 080.10.RO (15) | 0.245 | 0.073 | g |
| Strike-Through | WSP 070.3.RO (15) | 1.97 | 0.08 | second |
| time | | | | |
| Tenacity (CRE) | WSP 110.4.RO (15) | 5.87 | 0.55 | N/2.5 cm |
| Opt A | | | | |
| Elongation (CRE) | WSP 110.4.RO (15) | 9.38 | 2.15 | % |
| Opt A | | | | |

### Example 5

Two prototype acquisition distribution layer samples were independently tested by SGS in Wisconsin USA against a well performing commercial incontinence product for rate of acquisition and rewet. The testing was done in accordance with ISO 17025 accreditation. Prototype Sample 1 comprised 85% wool fibre and 15% viscose at a weight of 60 grams per square metre. Prototype Sample 2 comprised 80% wool and 20% Lenzing viscose (1.3 dtex, 38 mm fibre length) at a weight of 60 grams per square metre. To ensure comparisons between the commercial product and the prototypes, the acquisition distribution layer of the commercial product was removed and replaced with the respective acquisition distribution layer of Prototype 1 or Prototype 2. The results are shown below in Table 7.

TABLE 7

| | | | Prototype 1 | Prototype 2 |
|---|---|---|---|---|
| Property being | | Commercial | 85% wool: | 80% wool: |
| tested | | Product | 15% viscose | 15% viscose |
| Rate of | Average | 25 | 18 | 24 |
| Acquisition (s) | (seconds) | | | |
| | Standard | 2.8 | 1.4 | 3.5 |
| | Deviation | | | |
| | Maximum | 29 | 19 | 27 |
| | Minimum | 22 | 17 | 22 |
| | N = | 5 | 2 | 2 |
| | Insult | 75 | 75 | 75 |
| | Volume (ml) | | | |
| | Insult Rate | 7 | 7 | 7 |
| | (ml/sec) | | | |
| Rewet | Average | 0.0500 | 0.0820 | 0.0795 |
| | Standard | 0.0212 | 0.00000 | 0.01485 |
| | Deviation | | | |
| | Maximum | 0.0520 | 0.0820 | 0.0900 |
| | Minimum | 0.0470 | 0.0820 | 0.0690 |
| | N = | 5 | 2 | 2 |

### Example 6

It is also envisaged that a top sheet layer would be configured to have a weight of between about 15-50 grams per square metre, suitable for a diaper or hygiene article that sits next to the skin. The top sheet layer could have a similar composition to the Prototype Samples 1 and 2 described above in Example 5. It is anticipated that with a top sheet layer, the wool used could use a lower micron fibre, such as a 24-30 micron fibre, for extra softness.

It is also to be appreciated that a hygiene article could be configured comprising a combination of one or more hygiene articles. For example, a hygiene article could be configured comprising a top sheet layer and an acquisition distribution layer.

It is also to be appreciated that other hygiene articles, such as a non-woven wipe, facemask suitable material and an absorbent core for a range of absorbent purposes could be developed and configured from variants of Prototype Sample 1 or Prototype Sample 2. A person skilled in the art would be able to configure a desired hygiene article once the features and functional requirements of the hygiene article were known.

The disclosures of each and every patent, patent application, and publication cited herein are hereby incorporated herein by reference in their entirety. While this disclosure has been described with reference to specific aspects, it is apparent that other aspects and variations may be devised by others skilled in the art without departing from the true spirit and scope of the disclosure. The appended claims are intended to be construed to include all such aspects and equivalent variations. Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference.

While this disclosure has been particularly shown and described with references to preferred embodiments thereof, it will be understood by those skilled in the art that various changes in form and details may be made therein without departing from the scope of the disclosure encompassed by the appended claims.

1. A hygiene article including a fibre composition comprising a combination of sheep wool fibres and polymeric fibres.

2. The hygiene article as claimed in claim 1 wherein the sheep wool fibres are scoured wool fibres.

3. The hygiene article as claimed in claim 1 or claim 2, wherein the sheep wool fibres are between about 16-40 microns in thickness.

4. The hygiene article as claimed in any one of claims 1 to 3, wherein the polymeric fibres are sustainably produced and biodegradable or compostable.

5. The hygiene article as claimed in claim 4, wherein the polymeric fibres are between about 16-40 mm in length.

6. The hygiene article as claimed in any one of claims 1 to 4, wherein the polymeric fibres are selected from polylactic acid fibres, cellulosic polymeric fibres, milk fibres or combinations thereof.

7. The hygiene article as claimed in any one of claims 1 to 6, wherein the fibre composition comprises between about 20-99% wool fibres and between about 1-80% polymeric fibres.

8. The hygiene article as claimed in any one of claims 1 to 7, wherein the fibre composition comprises between about 20-97% wool fibres and between about 3-80% polymeric fibres.

9. The hygiene article as claimed in any one of claims 1 to 8, wherein the fibre composition comprises about 30-95% wool fibres and about 5-70% polymeric fibres.

10. The hygiene article as claimed in any one of claims 1 to 9, wherein the fibre composition further includes a fibre binding agent.

11. The hygiene article as claimed in any one of claims 1 to 6, wherein the fibre composition comprises between about 30-70% wool fibre and between about 70-30% polylactic acid fibres.

12. The hygiene article as claimed in any one of claims 1 to 6 and 11, wherein the fibre composition comprises between about 40-60% wool fibre and between about 60-40% polylactic acid fibres.

13. The hygiene article as claimed in any one of claims 1 to 6, 11 and 12, wherein the fibre composition comprises between about 50% wool fibre and about 50% polylactic acid fibres.

14. The hygiene article as claimed in any one of claims 1 to 6, wherein the fibre composition comprises between about 30-70% wool fibre and between about 70-30% cellulosic polymeric fibres.

15. The hygiene article as claimed in any one of claims 1 to 6 and 14, wherein the fibre composition comprises between about 30-45% wool fibre and between about 30-45% cellulosic polymeric fibres and between 10-45% of a fibre binder.

16. The hygiene article as claimed in any one of claims 1 to 6, 14 and 15, wherein the cellulosic polymeric fibres are viscose fibres.

17. The hygiene article as claimed in any one of claims 1 to 6, wherein the fibre composition comprises between about 20-99% wool fibres and between about 1-80% viscose fibres.

18. The hygiene article as claimed claim 17, wherein the fibre composition comprises between about 80-98% wool fibres and between about 2-20% viscose fibres.

19. The hygiene article as claimed in claim 18, wherein the fibre composition comprises between about 90-96% wool fibres and between about 4-10% viscose fibres.

20. The hygiene article as claimed in claim 19, wherein the fibre composition comprises about 95% wool fibres and about 5% viscose fibres.

21. The hygiene article as claimed in any one of claims 1 to 20, wherein the weight of the hygiene article is between about 50-70 gram per square meter.

22. The hygiene article as claimed in any one of claims 1 to 21, wherein the weight of the hygiene article is about 60 gram per square meter.

23. The hygiene article as claimed in any one of claims 1 to 23, wherein the thickness of the hygiene article is between about 0.5 to 2.0 mm.

24. The hygiene article as claimed in any one of claims 1 to 23, wherein the thickness of the hygiene article is between about 1.0 to 1.5 mm.

25. The hygiene article as claimed in any one of claims 1 to 24, wherein the thickness of the hygiene article is about 1.0 mm.

26. The hygiene article as claimed in any one of claims in 1 to 25, wherein the hygiene article is an acquisition distribution layer, a topping sheet or an absorbent core layer.

27. The hygiene article as claimed in claim 26, wherein the hygiene layer is an acquisition distribution layer.

28. A method of producing a hygiene article, as claimed in any one of claims 1 to 27, the method including the steps of:
(a) combining wool fibres and polymeric fibres together to provide a fibre composition; and
(b) bonding the combined fibres.

29. The method as claimed in claim 28, wherein the method includes the further step of applying a wetting agent to the fibre composition either before or after the bonding step.

30. The method as claimed in claim 28, wherein the wetting agent is Cirrasol 910XS-LQ (CRODA).

31. The method as claimed in any claim 29 or claim 30, wherein the method includes the further step of drying the fibre composition after the wetting agent has been applied.

32. The method as claimed in any one of claims 28 to 31, wherein the bonding step is a thermal bonding step carried out at about 130 degrees C.

33. The method as claimed in claim 32, wherein the thermal bonding step is carried out for about 1 minute.

34. The method as claimed in any one of claims 28 to 33, further including the step of adding a binder after the bonding step.

35. The method as claimed in any one of claims 28 to 31, wherein the bonding step is a mechanical bonding step.

36. The method as claimed in claim 35, wherein the mechanical bonding step is a hydroentanglement step.

37. The method as claimed in any one of claims 28 to 31, wherein the bonding step is a chemical bonding step using a binder.
